(19) United States
(12) Patent Application Publication
(10) Pub. No.: US 2023/0174569 A1
(43) Pub. Date: Jun. 8, 2023

(54) PROCESS PER PREPARING (3a,5a)-20-OXOPREGNAN-3-YL GLYCYL-L-VALINATE HYDROCHLORIDE

(71) Applicant: INDUSTIALE CHIMICA S.R.L., Milano (IT)

(72) Inventors: Roberto Lenna, S. Giorgio Su Legnano (IT); Andrea Fasana, Nessi (IT); Francesco Barbieri, Azzate (IT); Roberta Sinisi, Sumirago (IT); Stefania Vallese, Cardano Al Campo (IT)

(21) Appl. No.: 18/071,417

(22) Filed: Nov. 29, 2022

(30) Foreign Application Priority Data
Dec. 3, 2021 (IT) 102021000030671

(51) Int. Cl. C07J 41/00

(52) U.S. Cl. CPC C07J 41/005

(57) ABSTRACT

The present invention refers to a process for the preparation of (3α,5α)-20-oxopregnan-3-yl glycyl-L-valinate hydrochloride, a compound having the formula I reported below:

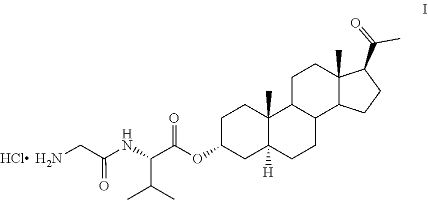

### FIELD OF THE INVENTION

The present invention relates to the sector of processes for the synthesis of active ingredients for pharmaceutical use, and in particular to a process for preparing (3a,5a)-20-oxopregnan-3-yl glycyl-L-valinate hydrochloride on an industrial scale. The compound is identified with CAS Number 2609098-69-1 and belongs to the class of Brexanolone derivatives. Such derivatives may be used in preparing drugs to prevent or treat disorders of the central nervous system, and according to what is reported in WO 2021/027744 A1, (3a,5a)-20-oxopregnan-3-yl glycyl-L-valinate hydrochloride has a better profile than Brexanolone in terms of water solubility, storage stability in aqueous solutions, and formulation.

### STATE OF THE ART

(3α,5α)-20-oxopregnan-3-yl glycyl-L-valinate hydrochloride has the structure reported below:

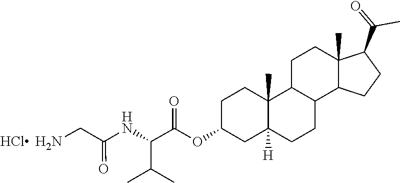

Patent application WO 2021/027744 A1 of Nanjing Noratech Pharmaceutical Co., LTD, describes the compound within a general formula (claim 1) and discloses the structure of the corresponding free base in claim 6.

In WO 2021/027744 A1 the synthesis of (3α,5α)-20-oxopregnan-3-yl glycyl-L-valinate hydrochloride (compound 2) is reported according to the following scheme:

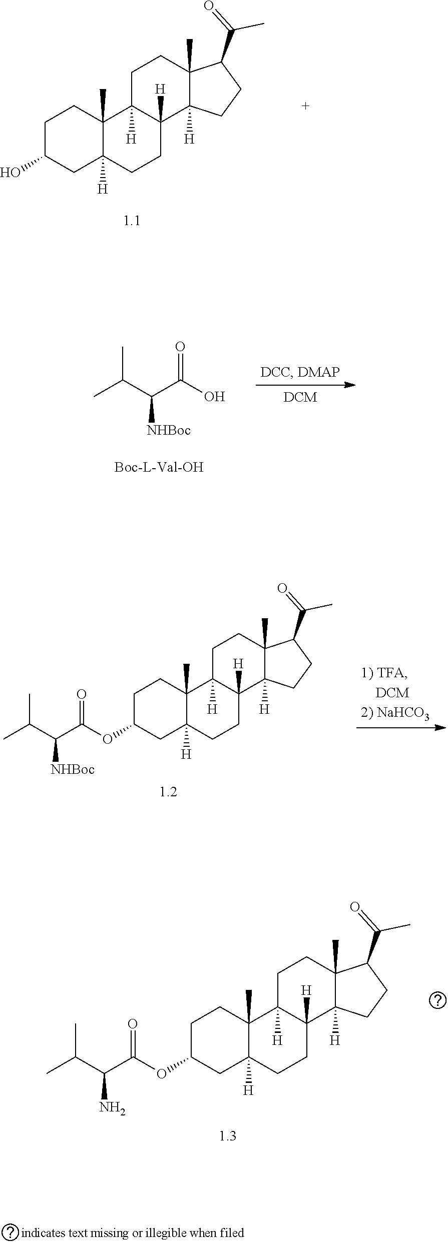

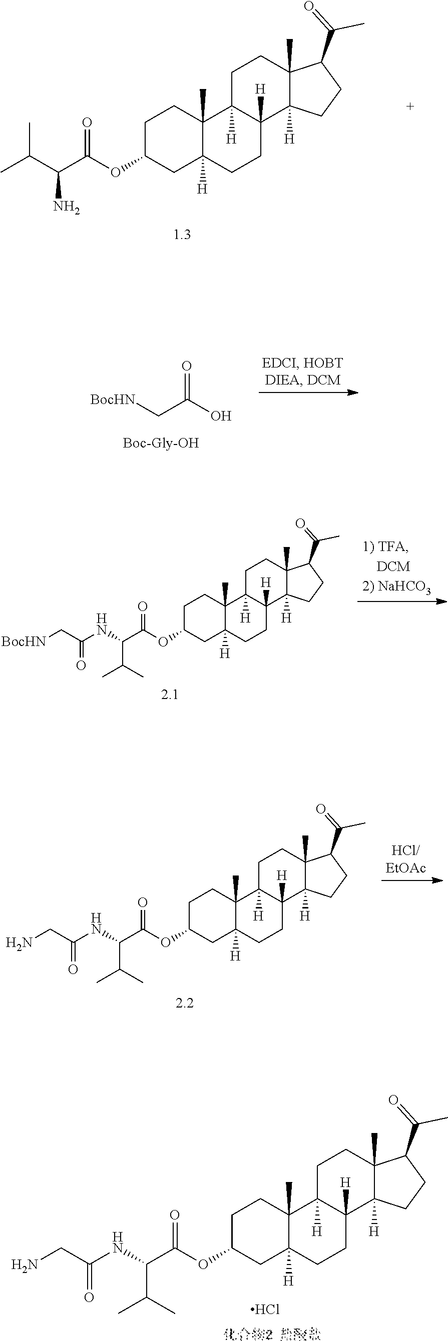

The starting compound 1.1 is Brexanolone.

WO 2021/027744 A1 reports that the starting Brexanolone used is commercially available or can be prepared according to a known method, which however is not specified in the text.

In the preparation of compound 2, the Brexanolone starting material is subjected to a linear synthesis consisting of 5 consecutive synthetic steps. The synthetic steps involve the step-by-step functionalization of Brexanolone, with the insertion of one amino acid at a time, followed by the deprotection thereof in acids, before carrying out the reaction with the next amino acid and further deprotection in acids. A further final step in acids is then carried out to convert the free base into the hydrochloride.

This process has obvious drawbacks, which make it inefficient from an industrial point of view.

A first drawback is a high starting steroid consumption. The commercial availability of Brexanolone on an industrial scale is limited, while the amino acids necessary for preparing the compound (3α,5α)-20-oxopregnan-3-glycyl-L-valinate 2.2 are widely available commercially.

A second drawback is that, out of five consecutive transformations from Brexanolone, compound 1.1, to (3α,5α)-20-oxopregnan-3-yl glycyl-L-valinate hydrochloride, compound 2, three of them subject the steroid skeleton to acidic conditions: two protective group removals and a transformation from free base to hydrochloride.

In patent application WO 2020/083839 A1, experimental evidence is reported of the instability of Brexanolone and its synthetic intermediates with a pregnanic structure when subjected to acidic conditions: the side reaction is the epimerization in position 17, which occurs through the formation of an enol intermediate during the course of the reaction:

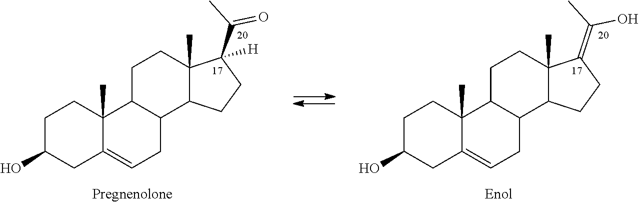 [SMILES: CC(=O)[C@H]1CCC2C3CC=C4C[C@@H](O)CC[C@]4(C)C3CC[C@@]21C ; C/C(O)=C1/CCC2C3CC=C4C[C@@H](O)CC[C@]4(C)C3CC[C@]12C]

The regeneration of the ketone form occurs with the predominant formation of the R epimer, but the amount of a epimer, even if minor, affects the quality of the obtained product for pharmaceutical purposes.

In the light of the above, an improved process and of simple industrial applicability for the synthesis of (3α,5α)-20-oxopregnan-3-yl glycyl-L-valinate hydrochloride is therefore needed.

### SUMMARY OF THE INVENTION

This object is achieved with the present invention, relating to a process for the synthesis of (3α,5α)-20-oxopregnan-3-yl glycyl-L-valinate hydrochloride (compound I) comprising the following steps:
a) reaction, in the presence of an activating agent, of Brexanolone, 3α-hydroxy-5α-pregnan-20-one, intermediate N-3 of the process, with a N-GP-glycyl-L-valine dipeptide of formula 2, wherein GP is an amine protecting group, to obtain the intermediate N-2, (3α,5α)-20-oxopregnan-3-yl N-GP-glycyl-L-valinate:

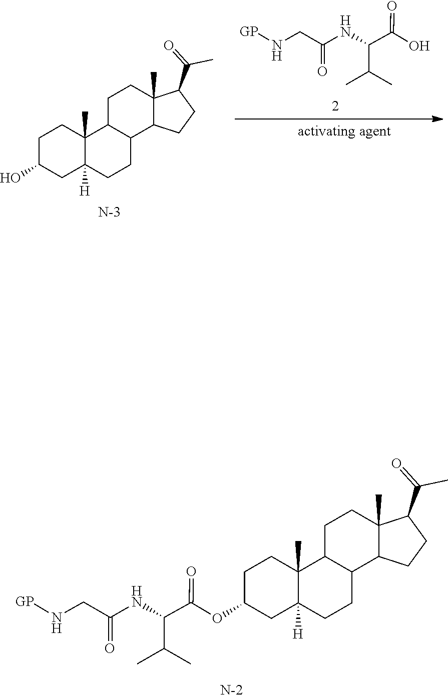

b) deprotection of intermediate N-2 and obtainment of compound I, according to one of the two alternative pathways b1) or b2) below:
   b1) in case the deprotection of intermediate N-2 is achieved without the use of hydrochloric acid:
   i) deprotection of intermediate N-2 to obtain intermediate N-1, (3α,5α)-20-oxopregnan-3-yl glycyl-L-valinate:

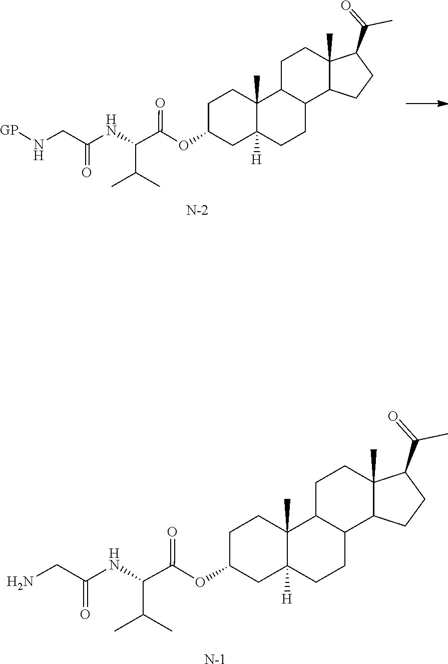

ii) treatment of intermediate N-1 with HCl in a solvent to obtain (3α,5α)-20-oxopregnan-3-yl glycyl-L-valinate hydrochloride, compound I:

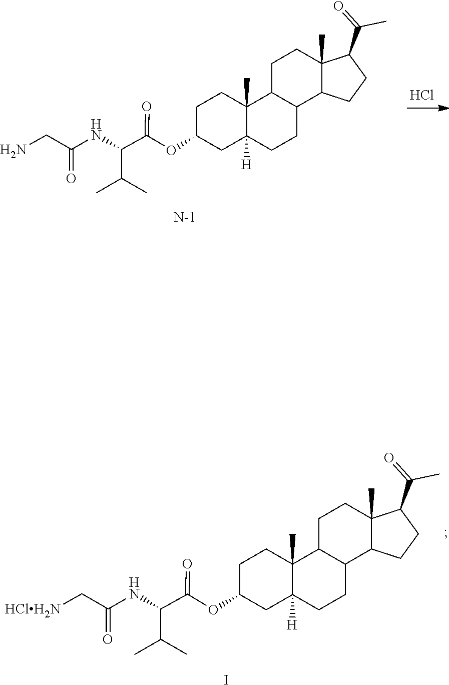

or
b2) deprotection of intermediate N-2 in the presence of hydrochloric acid to obtain (3α,5α)-20-oxopregnan-3-yl glycyl-L-valinate hydrochloride, compound I:

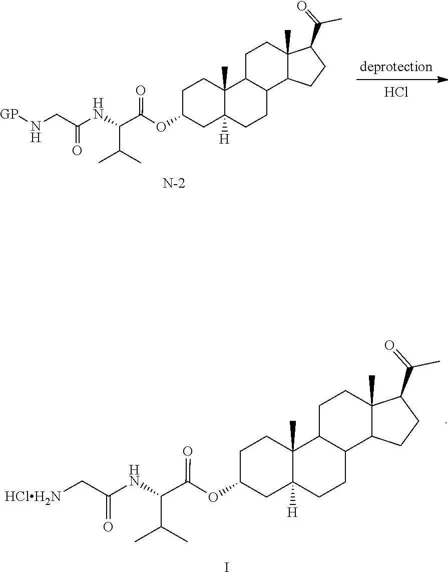

### DETAILED DESCRIPTION OF THE INVENTION

The invention relates to an improved process for the synthesis of (3α,5α)-20-oxopregnan-3-glycyl-L-valinate hydrochloride, compound I having the formula below, consisting of steps a) and b1) or b2) indicated above

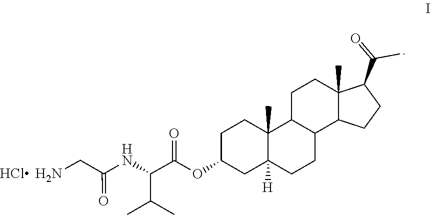

The process of the invention allows to limit the consumption of the steroid Brexanolone, by reducing the number of chemical transformations from Brexanolone to (3α,5α)-20-oxopregnan-3-glycyl-L-valinate through a convergent synthesis, and to reduce the number of reactions in an acidic environment, thus minimizing the undesired side reaction of epimerization of the obtained product.

In the description of the reactions that make up the process of the invention, the ratios between reactants are indicated as w/w, i.e. ratios by weight, unless otherwise specified.

Step a) consists in the reaction of Brexanolone, 3α-hydroxy-5α-pregnan-20-one, intermediate N-3, with the dipeptide of general formula N-GP-glycyl-L-valine, compound 2, in the presence of an activating agent, to give compound N-2:

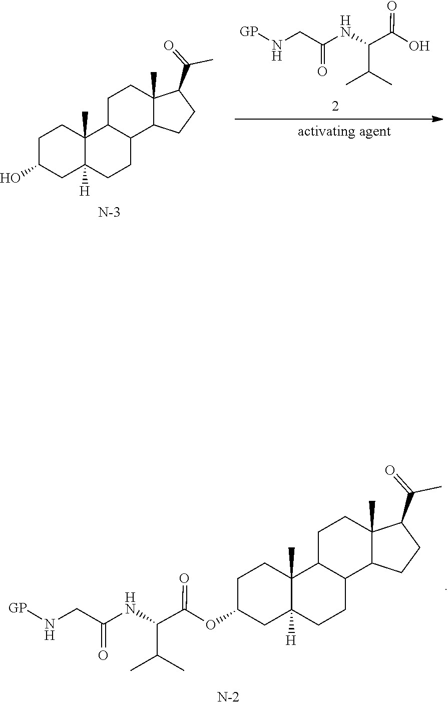

Brexanolone of suitable quality as the starting compound in step a) is commercially available or can be produced according to the teachings of WO 2020/083839 A1.

The compound 2 is glycyl-L-valine in which the —NH₂ group of the glycine radical is protected with an amine protective group. These protective groups are well known in organic chemistry and described by Greene et al. “Protective groups in Organic Synthesis”, John Wiley and Sons, 5th Edition, 2014.

The compound 2 is used in a molar ratio of between 1.75 and 3.5, preferably between 2.3 and 3.0, with respect to intermediate N-3.

The preferred amine protective group for the purposes of the invention is (1,1-dimethylethoxy)carbonyl (known by the acronym BOC); the compound that is reacted with Brexanolone is therefore preferably N-[(1,1-dimethylethoxy)carbonyl]glycyl-L-valine, compound 2′, to obtain intermediate N-2′ (3α,5α)-20-oxopregnan-3-yl N-[(1,1-dimethylethoxy)carbonyl]glycyl-L-valinate:

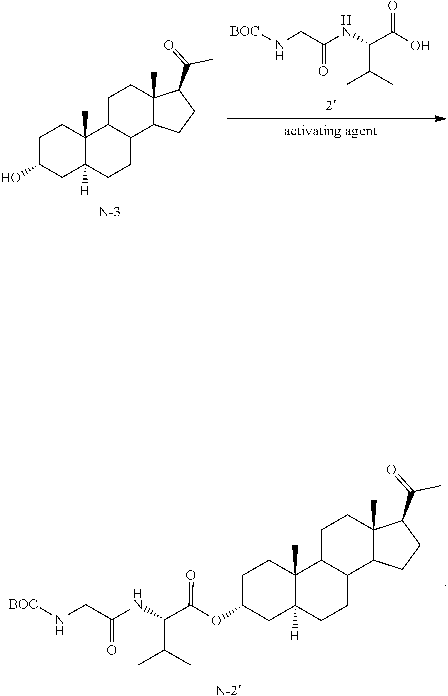

In the preferred case, i.e. use of compound 2′, this is used in a ratio (w/w) comprised between 1.5 and 3, preferably between 2.1 and 2.5, with respect to intermediate N-3.

Compound 2′, N-[(1,1-dimethylethoxy)carbonyl]glycyl-L-valine, CAS No. 28334-73-8, is commercially available.

Alternatively, compound 2′ can be prepared from L-Valine methyl ester hydrochloride by reaction with N-[(1,1-dimethylethoxy)carbonyl]glycine and subsequent hydrolysis, according to the following scheme:

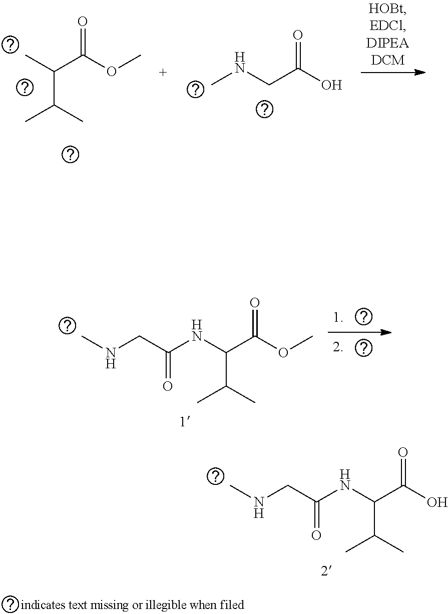

wherein intermediate 1′ can be prepared with the method described in Chem. Rev. 2011, 111, 6557, herein incorporated by reference, where HOBt=1-hydroxybenzotriazole, EDCl=1-ethyl-3-(3-dimethylamino-propyl)carbodiimide hydrochloride, DIPEA diisopropylethylamine, DCM=methylene chloride.

For the details of this synthesis, reference can be made to example 1 reported in the experimental part of the present description.

The activating agent for the activation of the carboxyl group is any one of the numerous compounds known for the purpose, such as carbodiimides, phosphonium salts such as benzotriazolyloxy-tris(dimethylamino), phosphonium hexafluorophosphate (BOP), 1H-benzotriazol-1-yloxytripyrrolidinophosphonium hexafluorophosphate (PyBOP) and the like, uronium salts such as 1H-benzotriazolium-1-[bis(dimethylamino)nethylene]-3-oxide hexafluorophosphate (HBTU), tetramethylamminium salts such as 1-[bis(dimethylamino)methylene]-1H-1,2,3-triazolo[4,5-b]piridinium 3-oxyde hexafluorophosphate (HATU), 1-(ethoxy carbonyl)-2-ethoxy-1,2-dihydroquinoline (EEDQ), or 1-methyl-2-chloropiridinium iodide (Mukaiyama reagent).

Preferred activating agents are carbodiimides, such as 1-ethyl-3-(3-dimethylaminopropyl)carbodiimide hydrochloride (EDCI), the corresponding free base (EDC), diisopropylcarbodiimide (DIC) or dicyclohexylcarbodiimide (DCC), in the presence of catalysts such as 4-dimethylaminopyridine (DMAP) or 1-hydroxybenzotriazole (HOBt).

For the present invention DCC is preferably used, in the presence of DMAP in catalytic amounts. In particular, DCC is used in a ratio (w/w) comprised between 1.3 and 2, preferably between 1.4 and 1.8, with respect to intermediate N-3, and DMAP is used in amounts comprised between 5 and 10% by weight, preferably 8% by weight, with respect to intermediate N-3.

The reaction is carried out in a solvent selected from chlorinated solvents such as dichloromethane (DCM) or tetrachloromethane (CCl₄), hydrocarbons such as benzene or toluene, ethers such as tetrahydrofuran (THF) or dioxane, dipolar aprotic solvents such as dimethyl sulfoxide (DMSO), dimethylformamide (DMF), acetonitrile, pyridine, and mixtures thereof. The preferred solvent is toluene.

Step a) is carried out at a temperature comprised between 50 and 80° C., preferably between 55 and 70° C., for a time comprised between 8 and 24 hours, preferably between 12 and 18 hours.

Step b) alternatively consists in the sequence of steps b1 i) and b1 ii), or in step b2) shown above.

Step b1 i) is the deprotection of intermediate N-2 to intermediate N-1, (3α,5α)-20-oxopregnan-3-yl glycyl-L-valinate:

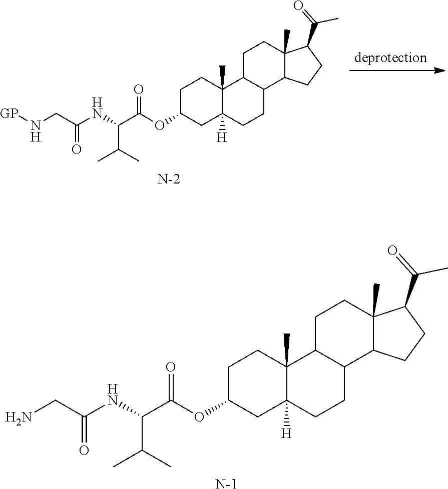

The reaction may be carried out with a reagent selected from a mineral acid, an organic acid, an acyl chloride, iodine, trimethylsilyl iodide, a secondary amine, or hydrogen, optionally followed by treatment with bases.

Step b1 ii) consists in the treatment of intermediate N-1 with HCl in a suitable solvent, to obtain (3α, 5α)-20-oxopregnan-3-glycyl-L-valinate hydrochloride, compound I:

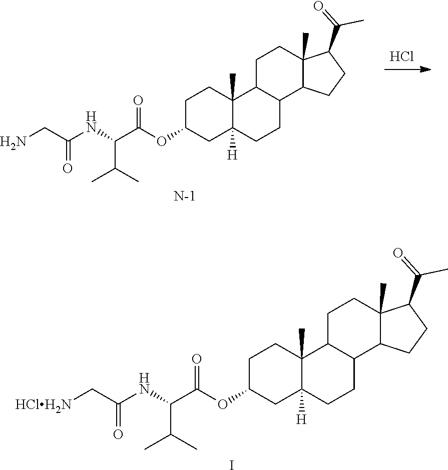

Useful solvents for this step are an ester selected from ethyl acetate (AcOEt) or isopropyl acetate (AcOiPr), or an alcohol such as methyl alcohol (MeOH), ethyl alcohol (EtOH) or isopropyl alcohol (i-PrOH).

In the preferred case of using compound 2′, N-[(1,1-dimethylethoxy)carbonyl]-glycyl-L-valine in forming intermediate N-2′, route b1) is preferably followed.

The removal of the BOC protective group (step b1 i) can be carried out using one of the following methods: trifluoroacetic acid in methylene chloride; HCl in organic solvents such as MeOH, ethyl acetate, 1,4-dioxane and acetone; acetyl chloride in MeOH; trimethylsilyliliodide in MeOH; catalytic iodine; or, preferably, oxalyl chloride in MeOH, as in the following scheme:

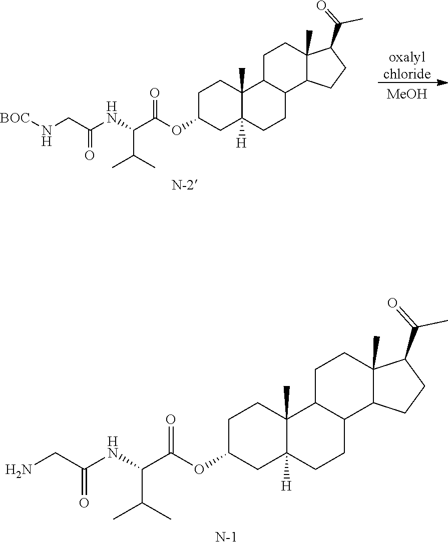

Oxalyl chloride is used in a ratio (w/w) comprised between 0.8 and 1.3, preferably between 0.9 and 1.1, with respect to intermediate N-2′.

The reaction is carried out by operating at a temperature comprised between 40 and 80° C., preferably between 50 and 70° C., for a time between 0.5 and 1.5 hours.

The formation of the hydrochloride (step b1 ii) is carried out using HCl in EtOAc:

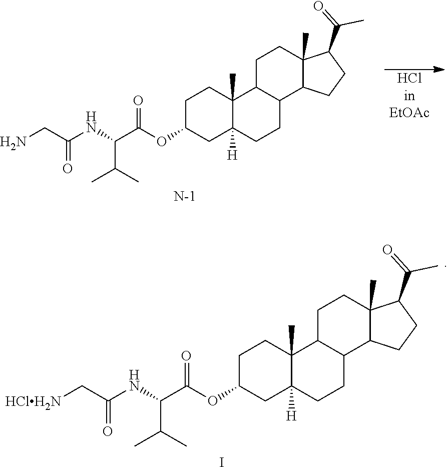

Hydrochloric acid is used in a w/w ratio between 2 and 4, preferably between 2.5 and 3, with respect to intermediate N-1.

The salt formation takes place at a temperature comprised between 0 and 35° C., preferably between 20 and 30° C., for a reaction time comprised between 0.5 hours and 4 hours, preferably between 1 and 3 hours.

Alternatively, step b) can be carried out through the single step b2) of deprotection of intermediate N-2 in the presence of hydrochloric acid:

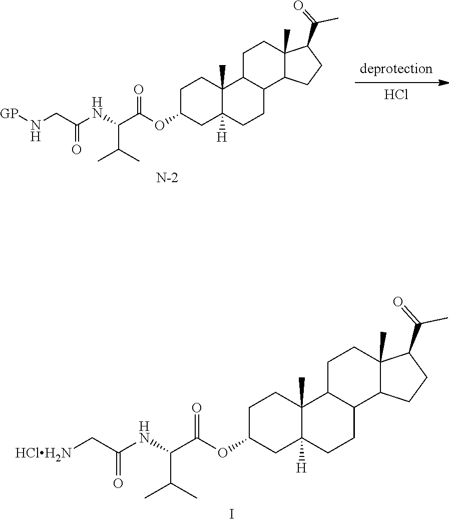

In the preferred case of using intermediate N-2′, the reaction is carried out in a solvent selected from MeOH, i-PrOH and, preferably, EtOH:

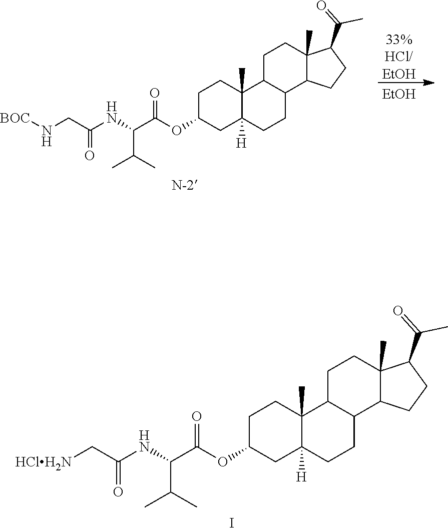

The reaction temperature is comprised between 50 and 70° C., for a time between 2 and 6 hours.

The invention will be further illustrated by the following examples.

Instruments, Methods and Experimental Conditions

Nmr:

NMR spectrometer JEOL 400 YH (400 MHz); Software JEOL Delta v5.1.1; Spectra recorded in deuterated solvents such as: Chloroform-d, D 99.8%, containing 0.1% (v/v) tetramethylsilane (TMS) as an internal standard; and Chloroform-d, “100%”, D 99.96%, containing 0.03% (v/v) TMS, and DMSO-d₆.

MS:

Instrument: DSQ-trace Thermofisher

Sample introduction—direct exposure probe (dep)

Chemical ionization (CI) with methane

Methane pressure: 2.2 psi

Source temperature: 200° C.

TLC:

MERCK: TLC Silica Gel 60 F254 Aluminium sheets 20×20 cm, cod. 1.0554.0001.

TLC Detection Reagent:

Cerium phosphomolybdate: 25 g of phosphomolybdic acid and 10 g of cerium(IV) sulfate are dissolved in 600 mL of H₂O. 60 mL of 98% H₂SO₄ are added and brought to a volume of 1

L with H₂O. The TLC plate is impregnated with the solution and then heated until the products are detected.

Notes

The water used in the experimental descriptions is to be intended as commercial distilled water, unless otherwise indicated.

The organic solvents used in the experimental descriptions are to be intended of a “technical” grade, unless otherwise indicated.

The reagents and catalysts used in the experimental descriptions are to be intended of commercial quality, unless otherwise indicated.

### Example 1

This example refers to the preparation of compound 2′ used in the process of the invention.

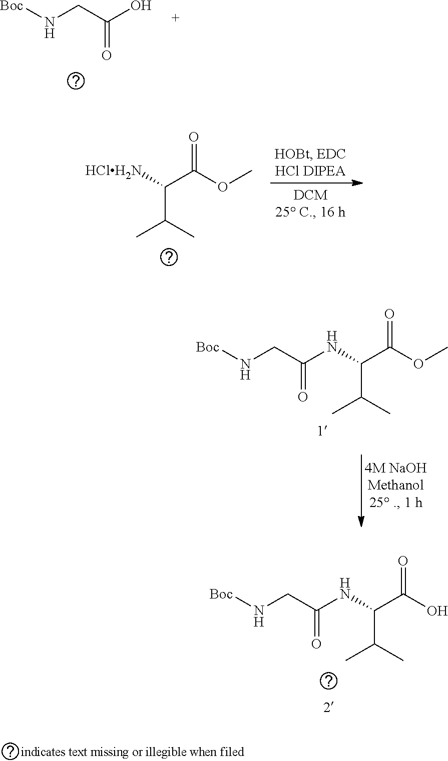

A flask is charged with 20 g of N-[(1,1-dimethylethoxy)carbonyl]glycine, 22.6 g of L-valine methyl ester hydrochloride, 85 mL of N,N-diisopropylethylamine and 500 mL of dichloromethane.

27.7 g of hydroxybenzotriazole are added and the solution is cooled to −15° C. for 15 minutes.

39.4 g of 1-ethyl-3-(3-dimethylaminopropyl) carbodiimmide hydrochloride (EDCI) are added and the mixture is kept under stirring at 25° C. for 16 hours.

Once the reaction is complete (TLC analysis) the reaction mixture is poured into 400 mL of saturated sodium bicarbonate solution, the layers are separated, and the aqueous layer is re-extracted with dichloromethane.

The organic layer is washed with a saturated sodium chloride solution and concentrated under reduced pressure at 45° C. to obtain 47 g of yellow oil.

The product is purified using silica gel column chromatography eluting with a 70:30 ethyl acetate/heptane mixture. The solvent is concentrated under reduced pressure at 45° C. to obtain 29.8 g of colorless oil, compound

The residue is taken up with 450 mL of MeOH, the solution is cooled to 5° C. and a 4M sodium hydroxide solution (450 mL) is slowly added. The mixture is kept under stirring at 25° C. for 1 hour.

Once the reaction is complete (TLC analysis) the solvent is concentrated by distillation under reduced pressure at 45° C.

The residue is taken up with ethyl acetate (200 mL) and brought to pH 5 with 37% hydrochloric acid.

The layers are separated and the solvent is concentrated under reduced pressure at 45° C. to obtain 28.6 g of crude N-[(1,1-dimethylethoxy)carbonyl]glycyl-L-valine.

The crude product is suspended in 170 mL of water and 30% sodium hydroxide is added up to basic pH. The aqueous layer is washed twice with ethyl acetate (2×100 mL) and the layers are separated.

The aqueous layer is acidified with 37% hydrochloric acid and extracted twice with ethyl acetate (2×100 mL). The organic layer is concentrated under reduced pressure at 45° C. to obtain 16.9 g of N-[(1,1-dimethylethoxy]carbonyl]glycyl-L-valine (white solid), compound 2′, whose ¹H-NMR, ¹³C-NMR and MS analytical data coincide with those reported in the literature.

¹H-NMR (400 MHz, DMSO-d₆): 12.71 (s, 1H); 7.79 (d, 1H, J=8.6 Hz); 6.99 (t, 1H, J=5.9 Hz); 4.18-4.15 (m, 1H); 3.58 (d, 2H, J=6.0 Hz); 2.01-2.8 (m, 1H); 1.38 (s, 9H); 0.85-0.87 (m, 6H).

¹³C-NMR (400 MHz, DMSO-d₆): 172.9; 169.4; 155.8; 78.0; 56.9; 43.0; 30.1; 28.2; 19.0; 17.8.

Mass (CI): m/z=275 [M⁺+1]; 175 [M⁺+1-Boc].

### Example 2

This example refers to the reaction for forming intermediate N-2′.

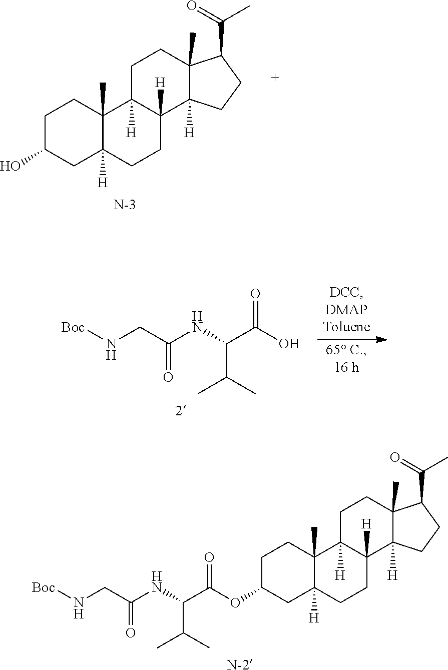

A flask is charged with 2.5 g of Brexanolone (intermediate N-3), 5.4 g of compound 2′ and 25 ml of toluene. 0.2 g of 4-dimethylaminopyridine and a solution of N,N′-dicyclohexylcarbodiimide (4.0 g) in toluene (4 mL) are added.

The mixture is brought to 65° C. and kept under stirring for 16 hours.

Once the reaction is complete (TLC analysis) the solid is filtered and the filtration liquid is concentrated by distilling under reduced pressure at 45° C. to obtain 8.1 g of crude intermediate N-2′ (yellow solid).

The product is purified using silica gel column chromatography eluting with a 50:50 heptane/ethyl acetate mixture.

The solvent is concentrated under reduced pressure at 45° C. to obtain 3.1 g of intermediate N-2′ (white solid).

The intermediate N-2′ obtained, subjected to ¹H-NMR, ¹³C-NMR and MS analysis shows the following analytical data:

¹H-NMR (400 MHz, DMSO-d₆): 7.93 (d, 1H, J=8.8 Hz); 6.95 (q, 1H, J=11.8/6 Hz); 4.94-4.92 (m, 1H); 4.21-4.17 (m, 1H); 3.62-3.59 (m, 2H); 2.56 (t, 1H, J=8.8 Hz); 2.03 (s, 3H); 2.06-1.08 (m, 22H); 1.33 (s, 9H); 0.85-0.86 (m, 7H); 0.76 (s, 3H); 0.51 (s, 3H).

¹³C-NMR (400 MHz, DMSO-d₆): 209.10; 171.32; 170.14; 156.31; 78.51; 70.70; 63.22; 57.77; 56.39; 54.04; 44.05; 43.42; 40.10; 38.63; 35.93; 35.45; 33.80; 33.07; 32.81; 32.12; 31.98; 31.72; 30.73; 28.70; 28.35; 26.06; 24.44; 22.71; 20.88; 19.41; 18.41; 13.69; 11.58.

Mass (CI): m/z=475 [M⁺+1-Boc].

### Example 3

This example refers to the obtainment of compound I starting from intermediate N-2′ according to route b1).

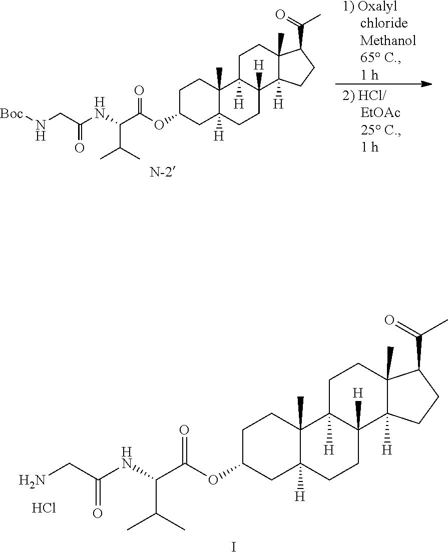

A flask is charged with intermediate N-2′ (1 g) obtained according to the procedure described in the previous example and 60 mL of methanol. The mixture is kept under stirring at 25° C. for 10 minutes. 1 g of oxalyl chloride is added to the solution and it is heated at 65° C. for 1 hour.

Once the reaction is complete (TLC analysis) the solvent is eliminated by distilling under reduced pressure at 45° C. until 1.3 g of yellow solid are obtained.

The product is purified using silica gel column chromatography eluting with a 95:5 methylene chloride/MeOH mixture. The solvent is concentrated under reduced pressure at 45° C. to obtain 0.6 g of white solid.

The residue is taken up with 4 mL of ethyl acetate, a 19.5% solution of hydrochloric acid in ethyl acetate (9 mL) is added dropwise and the mixture is kept under stirring at 25° C. for 1 h.

The solvent is concentrated to a small volume (1 mL) and slowly added dropwise to an isopropyl ether solution (20 mL) put under stirring at 25° C.

The suspension is kept under stirring at 25° C. for 1 hour and the solid is filtered by washing with isopropyl ether.

The solid is dried under reduced pressure at 45° C. to obtain 0.4 g of the desired compound I, (3α,5α)-20-oxopregnan-3-yl glycyl-L-valinate hydrochloride, as a white solid whose ¹H-NMR and Ms analytical data coincide with those reported in the literature.

¹H-NMR (400 MHz, CDCl₃): 8.28 (m, 1H); 8.16 (bs, 3H); 5.14-5.03 (m, 1H); 4.55 (d, 1H, J=4.2 Hz); 4.27 (d, 1H, J=8.8 Hz); 4.10 (d, 1H, J=8.8 Hz); 2.52 (t, 1H, J=8.8 Hz); 2.11 (s, 3H); 2.26-1.08 (m, 22H); 0.89-1.08 (m, 7H); 0.79 (s, 3H); 0.61 (s, 3H).

Mass (CI): m/z=475 [M⁺+1]

### Example 4

This example refers to the obtainment of compound I starting from intermediate N-2′ according to route b2).

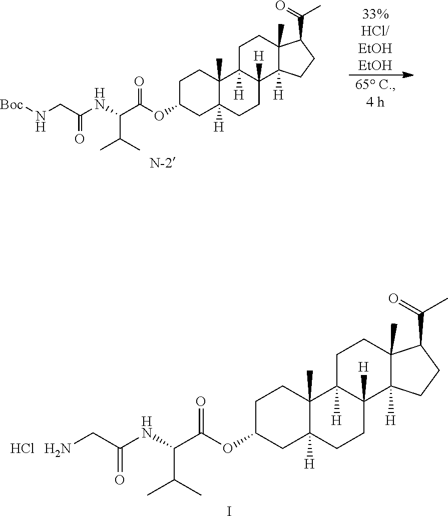

A flask is charged with intermediate N-2′ (1 g) obtained according to the procedure described in example 2 and 10 mL of EtOH. A 33% hydrochloric acid solution in EtOH (0.4 mL) is added to the reaction mixture and it is heated at 65° C. for 4 hours.

Once the reaction is complete (TLC analysis) the solvent is eliminated under reduced pressure at 45° C. and the residue is taken up with 10 mL of ethyl acetate. Charcoal (0.1 g) and dicalite (0.1 g) are added to the mixture and it is kept under stirring at 25° C. for 30 minutes.

The solution is filtered and concentrated to a small volume (1 mL). It is slowly added dropwise to an isopropyl ether solution (15 mL) put under stirring at 25° C.

The mixture is kept under stirring at 25° C. per 30 minutes and the solid is filtered washing with isopropyl ether.

The solid is dried under reduced pressure at 50° C. to obtain 0.6 g of compound I (white solid) whose ¹H-NMR, ¹³C-NMR and MS analytical data coincide with those reported in the previous example.

1. A process for the synthesis of (3α,5α)-20-oxopregnan-3-yl glycyl-L-valinate hydrochloride (compound I), comprising the following steps:
a) reaction, in the presence of an activating agent, of Brexanolone, 3α-hydroxy-5α-pregnan-20-one, intermediate N-3 of the process, with a N-GP-glycyl-L-valine dipeptide of formula 2, wherein GP is an amine protecting group, to obtain the intermediate N-2, (3α,5α)-20-oxopregnan-3-yl N-GP-glycyl-L-valinate: 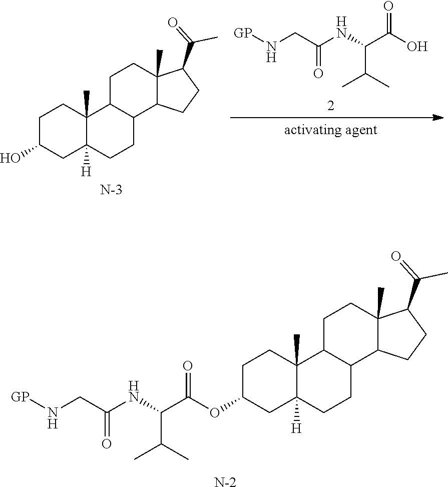
b) deprotection of intermediate N-2 and obtainment of compound I, according to one of the two alternative pathways b1) or b2) below:
b1) in case the deprotection of intermediate N-2 is achieved without the use of hydrochloric acid:
i) deprotection of intermediate N-2 to obtain intermediate N-1, (3α,5α)-20-oxopregnan-3-yl glycyl-L-valinate: 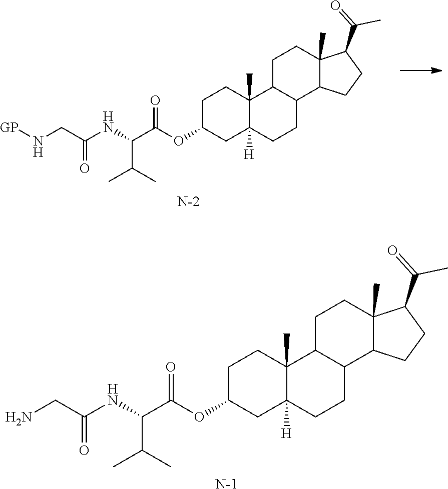
ii) treatment of intermediate N-1 with HCl in a solvent, to obtain (3α,5α)-20-oxopregnan-3-yl glycyl-L-valinate hydrochloride, compound I: 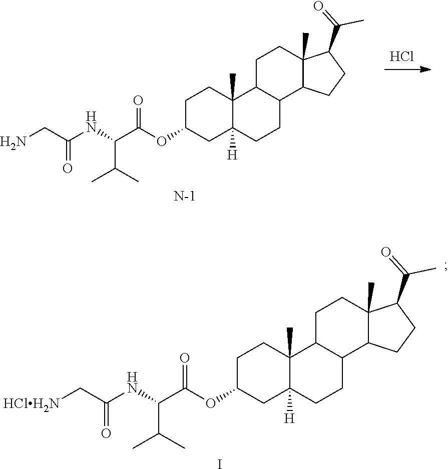
or
b2) deprotection of intermediate N-2 in the presence of hydrochloric acid to obtain (3α,5α)-20-oxopregnan-3-yl glycyl-L-valinate hydrochloride, compound I:

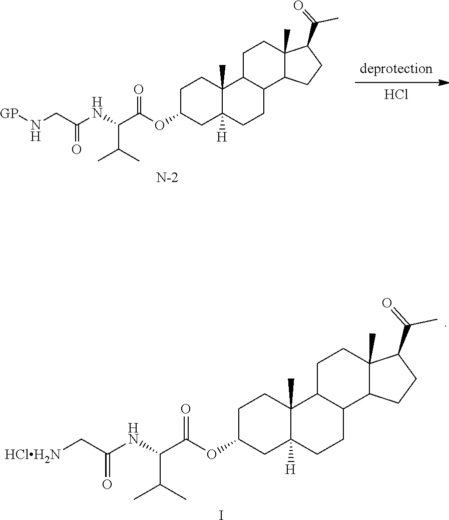

2. A process according to claim 1, wherein the dipeptide of formula 2 is N-[(1,1-dimethylethoxy)carbonyl]glycyl-L-valine, compound 2′:

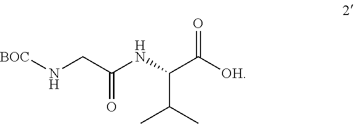 [SMILES: CC(C)[C@H](NC(=O)CNC(=O)OC(C)(C)C)C(=O)O]

3. A process according to claim 1, wherein compound 2 is used in a molar ratio of between 1.75 and 3.5 with respect to intermediate N-3.

4. A process according to claim 1, wherein the activating agent used in step a) is selected from carbodiimides, phosphonium salts, uronium salts, tetramethylaminium salts, 1-(ethoxycarbonyl)-2-ethoxy-1,2-dihydroquinoline (EEDQ) and 1-methyl chloropyridinium iodide (Mukaiyama reagent), and said step is carried out in the presence of a catalyst selected from 4-dimethylaminopyridine (DMAP) and 1-hydroxybenzotriazole (HOBt).

5. A process according to claim 4, wherein step a) is carried out with dicyclohexyl carbodiimide (DCC) as an activating agent in a ratio (w/w) of between 1.3 and 2 with respect to intermediate N-3, in the presence of 4-dimethylaminopyridine (DMAP) as a catalyst in an amount comprised between 5 and 10% by weight with respect to intermediate N-3.

6. A process according to claim 1, wherein step b1 i) is carried out with a reagent selected from a mineral acid, an organic acid, an acyl chloride, iodine, trimethylsilyliodide, a secondary amine and hydrogen.

7. A process according to claim 1, wherein step b1 ii) is carried out with HCl in a solvent selected from ethyl acetate, isopropyl acetate, methyl alcohol, ethyl alcohol and isopropyl alcohol.

8. A process according to claim 2, wherein compound 2′ is used in step a), and step b) is carried out according to pathway b1) using oxalyl chloride in methyl alcohol in step b1 i).

9. A process according to claim 2, wherein step b2) is carried out with HCl in a solvent selected from methyl alcohol, ethyl alcohol and isopropyl alcohol.
